(19) United States
(12) Patent Application Publication
(10) Pub. No.: US 2025/0143558 A1
(43) Pub. Date: May 8, 2025

(54) DENTAL VISION SYSTEM

(71) Applicant: A-dec, Inc., Newberg, OR (US)

(72) Inventors: Christopher C. Stone, Newberg, OR (US); Brett Trevor, Newberg, OR (US); Robert Michael Ayers, Newberg, OR (US); Jeffrey A. Zawada, Newberg, OR (US)

(21) Appl. No.: 18/386,959

(22) Filed: Nov. 3, 2023

(51) Int. Cl. A61B 1/24; A61B 1/00; A61B 1/045; A61B 1/06; G02B 27/00; G02B 27/01; G06T 3/60

(52) U.S. Cl. CPC A61B 1/24; A61B 1/000095; A61B 1/00016; A61B 1/00048; A61B 1/045; A61B 1/0684; G02B 27/0093; G02B 27/0172; G06T 3/60; A61B 2562/0219; A61B 2562/028; G02B 2027/0118; G02B 2027/0141; G02B 2027/0178

(57) ABSTRACT

A dental vision system comprises an intraoral camera component positionable within an oral cavity of a patient and movable manually by a user to obtain patient image data from various viewpoints within the oral cavity. The intraoral camera component has at least two camera modules and an inertial accelerometer configured to collect position and movement data relating to the intraoral camera component. A camera controller linked to the intraoral camera component receives the patient image data and the position and movement data relating to the intraoral camera component and is configured to determine a selected viewing orientation for the patient image data based at least in part on the position and movement data.

### FIELD

This application relates to dental instruments and devices, and in particular, to dental vision systems.

### BACKGROUND

Providing dentists and other practitioners with high visibility into a patient's oral cavity (i.e., the patient's mouth) is important to ensure that examination and dental procedures are effective. Conventionally, dentists seeking greater visibility into the oral cavity will bend and turn at the neck, back and waist, repositioning themselves to gain an adequate slightly different view. These repeated movements and positions, however, are often not ergonomically sound. They can lead to dentists experiencing discomfort and even developing chronic conditions, such as chronic neck and back pain, over time.

Dentists make use of specialized lighting, mirrors and loupes, among other devices, to improve their efforts to see into and visualize the patient's oral cavity. Video cameras are also used to capture images of the oral cavity, either from a position outside of the oral cavity (such as mounted to an overhead dental examination light) or from an intraoral camera maneuvered within the oral cavity by the dentist. Current camera offerings, however, do not meet all needs of providing a robust, full-featured solution to visualizing the oral cavity and communicating the image information to the dentist and others.

### SUMMARY

Described below are implementations of dental vision systems and associated methods that address shortcomings in the prior art.

According to one implementation, a dental vision system comprises an intraoral camera component positionable within an oral cavity of a patient and movable manually by a user to obtain patient image data from various viewpoints within the oral cavity, the intraoral camera component having at least two camera modules and an inertial accelerometer configured to collect position and movement data relating to the intraoral camera component, and a camera controller linked to the intraoral camera component, wherein the camera controller component receives the patient image data and the position and movement data relating to the intraoral camera component and is configured to determine a selected viewing orientation for the patient image data based at least in part on the position and movement data.

The camera controller can be programmed to apply an angular rotation to the patient image data such that each patient image is substantially aligned with the selected viewing orientation. The selected viewing orientation can include a gravitational up orientation. The selected viewing orientation can be determined with reference to a magnetic compass. The selected viewing orientation can be determined with reference to an absolute three-dimensional coordinate system.

According to another implementation, a dental vision system comprises an intraoral camera component positionable within an oral cavity of a patient, the intraoral camera comprising at least two video camera modules that are spaced apart from each other having respective fields of view that overlap each other, a plurality of LEDs positioned around the at least two camera modules, at least one baffle positioned between one of the at least two video camera modules and a respective outwardly adjacent group of the plurality of LEDs, the at least one baffle comprising a wall extending outwardly to reduce unwanted optical effects in images obtained by the at least two video camera modules.

The least one baffle extends outwardly to a baffle height higher than a height of the one of the at least two video camera modules. The at least one baffle can have a distal end, and wherein the distal end is configured to support a transparent barrier film applied to cover the at least two video camera modules, wherein the wall of the baffle is configured to block light rays emanating from the outwardly adjacent group of LEDs and reflected from the transparent barrier film to prevent the light rays from interfering with the images taken by the video camera modules. In some implementations, the video camera modules are mounted to a common plane.

According to another implementation, a dental vision system comprises an intraoral camera component positionable within an oral cavity of a patient and movable manually by a user to obtain patient image data from various viewpoints within the oral cavity and a head-worn display with at least one viewing area configured to selectively display the patient image data from the intraoral camera to a wearer of the head-worn display, wherein a location and size of the patient image data to be displayed are user selectable to improve visualization of the patient image data by the wearer.

The optical gradation in the viewing area can be reconfigured in multiple steps from opaque to transparent. The head-worn display can comprise a liquid crystal film selectively changeable to change optical gradation in the viewing area. The liquid crystal film can be within or applied to a lens of the head-worn display.

The viewing area of the head-worn display is configurable to change an optical gradation in the viewing area to improve visualization of the patient image data by the wearer.

The head-worn display can comprise a spectacle lens pair including a first spectacle lens and a second spectacle lens, and wherein the at least one viewing area comprises a portion of the total viewing area of at least one of first spectacle lens and the second spectacle lens. At least a portion of the spectacle lens pair can comprise a direct vision viewing area that is not configured to selectively display the patient image data.

The dental vision system can further comprise a camera controller linked to the intraoral camera component and the head-worn display, wherein the camera controller component receives the patient image data and is configured to transmit the patient image data wirelessly to the head-worn display.

The head-worn display can be configured to display 3D patient image data. According to another implementation, a dental vision system comprises an intraoral camera component positionable within an oral cavity of a patient and movable manually by a user to obtain patient image data from various viewpoints within the oral cavity and a head-worn display with at least one viewing area configured to selectively display the image data from the intraoral camera, further comprising a pupil tracking module configured to track at least one pupil of a wearer of the head-worn display to assist in controlling the display of the image data on the at least one viewing area.

The pupil tracking module can be configured to track a position of the at least one pupil of the wearer. The pupil tracking module can be configured to track a rate of movement of the at least one pupil of the wearer. The pupil tracing module can be configured to track dwell of the at least one pupil of the wearer that exceeds a predetermined time.

According to another implementation, a dental vision system comprises an intraoral camera component positionable within an oral cavity of a patient and movable manually by a user to obtain patient video image data from various viewpoints within the oral cavity, and an image stabilizer configured to process the patient video image data in raw form and generate stabilized patient video image data to reduce noticeable motion in the patient video image data.

The image stabilizer can be configured to address jitter in the patient video image data, including small displacements in frequency from 1 Hz to 60 Hz with a dimensional amplitude greater than one pixel in a horizontal extent, a vertical extent or combined horizontal and vertical extents. The image stabilizer can be configured to address blur in the patient video image data, including small displacements at a rate of change of less than 11 Hz with an amplitude of greater than one pixel.

The intraoral camera component can comprise at least one camera module and at least one MEMS accelerometer positioned relative to the at least one camera module to detect 6-axis acceleration of the camera module along translational and rotational axes.

The dental vision system can include a camera controller, wherein the camera controller receives the 6-axis acceleration data in an output signal from the MEMS accelerometer and generates pixel displacement and pixel displacement rate of change values correlated with a clock signal to apply to the patient video image data to produce a corrected patient video image for display.

The dental vision system can comprise an image processor configured to remove transient artifacts occurring in the oral cavity substantially in real time to improve image quality.

The foregoing and other objects, features, and advantages will become more apparent from the following detailed description, which proceeds with reference to the accompanying figures.

### BRIEF DESCRIPTION OF THE DRAWINGS

FIG. 1 is a perspective view of an exemplary dental vision system having an intraoral camera, a camera controller, a computer and a head-worn display.

FIG. 2 is perspective view of a dental unit control head showing the intraoral camera occupying one of the handpiece positions.

FIG. 3 is a perspective view of the intraoral camera of FIGS. 1 and 2.

FIG. 4 is an assembly view of the intraoral camera of FIGS. 1 and 2 showing its upper and lower housings separated from each other.

FIG. 5 is a perspective view of a prototype circuit board component for an intraoral camera.

FIG. 6 is another perspective view of the circuit board component of FIG. 5, also showing the overlapping fields of view of two camera modules.

FIG. 7 is an enlarged view of a distal end of the intraoral oral camera showing the camera modules and LEDs relative to openings and baffles in the housing.

FIG. 8 is a sectional view of the distal end of a housing without a baffle and showing a path of a typical light ray.

FIG. 9 is a sectional view similar to FIG. 8, except showing the distal end of the housing of FIG. 7 with one or more baffles are provided to change the effect of the typical light ray.

FIG. 10 is a block diagram of a system including a computing device for implementing the dental vision system.

FIG. 10B is a block diagram of a camera controller implemented as a standalone camera controller.

FIG. 11 is a representative image captured from video showing a tooth and a portion of an instrument where numerous spray droplets and/or spatter degrade the image quality.

FIG. 12 is an image corresponding to FIG. 11 after real-time video software enhancement has been applied to remove transient artifacts.

FIG. 13 is a view of a head-worn display configured for use in a direct vision mode.

FIG. 14 is a view of the head-worn display configured for use in an intraoral camera mode.

FIG. 15 is a schematic view of the user changing the viewing area size and location within the head worn display.

FIG. 16 is a representative screen display showing images from a patient's oral cavity being shared with others in a video conference setting.

FIG. 17 is a schematic depiction of teaching environment in which the images from the patient's oral cavity are projected to in-person attendees.

### DETAILED DESCRIPTION

Improving visibility into the oral cavity for dentists is a priority. Having clear vision of the oral cavity, from many different angles, is important to carrying out thorough and accurate examinations and procedures. Conventionally, dentists rely on moving their bodies to position their head and eyes for viewing into the patient's oral cavity, but this requires the dentist to repeatedly bend and turn at the neck, back and waist. Such movements are not ergonomically sound. Dentists frequently experience discomfort and even chronic problems over time.

The dental vision systems described herein provide better visibility of the oral cavity without requiring poor posture and contorted movements. Also, the dental vision systems enhance the dentist's vision in a number of ways. An intraoral camera enables viewing of the oral cavity from nearly any angle in real time. The image information can be displayed for the dentist as well as for the patient (such as to assist in explaining a treatment). The intraoral camera can be controlled to provide magnified and/or enhanced images that extend the dentist's native vision. The images from the intraoral camera can be oriented for proper viewing even as the camera is translated and/or rotated (e.g., using autorotation). Image stabilization can be applied to eliminate the effects of involuntary movement while holding and moving the intraoral camera.

Contamination of the intraoral camera and cross-contamination between patients can be prevented by using a single-use barrier with the intraoral camera that has a transparent area through which the camera modules operate. The intraoral camera housing can be designed with baffles that prevent stray reflections and other unwanted optical effects from occurring during use with the barrier.

The dental vision systems can also include a head-worn display. The head-worn display can be configured to display the image information from the intraoral camera to the wearer on display areas of one or both lenses of the display. Other non-display areas of the lenses allow for direct through vision. The display areas can be changed from opaque (for a display mode) to transparent (for a direct vision mode). Also, the display areas can be selectively changed in size and position within the lens. Eye tracking can be used to control changing the size and position of the display areas, as well as for controlling other operations and settings.

In examples where right and left display areas are used, techniques can be employed to present the image information to the wearer as 3D images. Images of the patient's teeth viewed in the display can be overlaid with corresponding X-ray images, cone beam CT images and other types of images for comparison. For other 3D images of the oral cavity, such as cone beam CT images or topographical models from a 3D scanner, the overlaid images can be configured to align with the orientation of the displayed images. Digital techniques can be employed to remove droplets and spatter from images so the subject matter is not obscured.

The dental vision system can be used in education settings to communicate image information from a patient, as seen by a dentist, to participants attending in person or via a video conference. Also, the wearer of the head-worn display can receive guided remote troubleshooting assistance, e.g., to evaluate an issue with an instrument or equipment in the dental operatory. These and other features are described in more detail below.

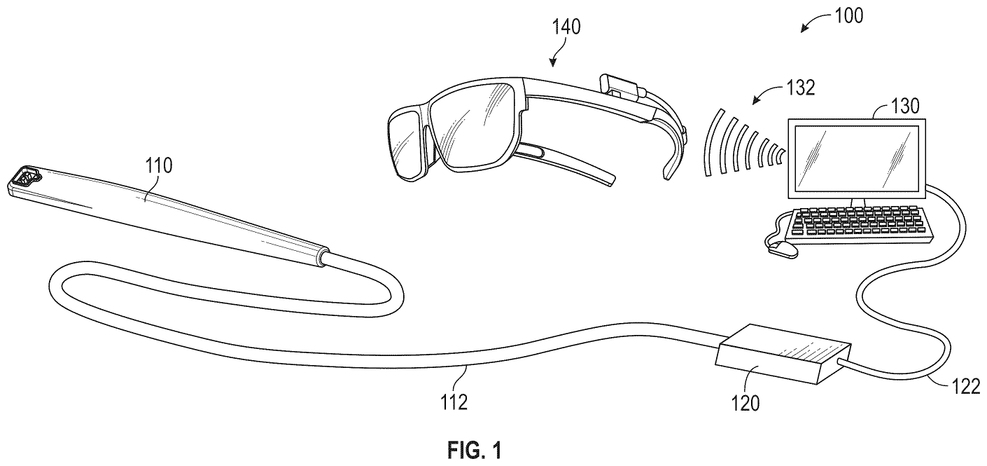

Referring to FIG. 1, a representative dental vision system 100 used to visualize a patient's oral cavity is shown. The dental vision system 100 includes an intraoral camera 110 designed to be held by a user and inserted into the patient's oral cavity during examination and/or treatment. The user manipulates the intraoral camera 110 by hand to allow images of areas of interest within the oral cavity to be captured.

The intraoral camera 110 is preferably connected to a camera controller 120 (which is a separate component in the dental vision system 100, but need not be). The camera controller 120 processes image data received from the intraoral cavity 110 and conveys signals or messages, such as commands or status, to and from the camera. The camera controller 120 is connected to the intraoral camera by a connection 112. In the illustrated implementation, the connection 112 is a hardwired cable connection, but a wireless connection between the intraoral camera and camera controller 120 is also possible.

The camera controller 120 manages camera functions, including but not limited to gain, exposure control and frame rate. Additionally, image orientation and stabilization and illumination intensity are managed by the camera controller 120.

The camera controller 120 receives a digital image(s) in a raw format and converts it to a standard format that can be displayed by most computers and other computing devices. The camera controller is the downstream data transceiver that de-modulates the image signal that is modulated by the camera interface in the handpiece. In some implementations, the cable 112 can have a small diameter and be highly flexible because it is communicating a demodulated image signal. The intraoral camera 110 can be supplied with power from a battery pack in the camera controller 120 or another source of electrical power connected to the camera controller 120 (e.g., a computer 130).

The camera controller 120 is connected to a computer 130 by a connection 122. In the illustrated implementation, the connection 122 is a hardwired cable connection, but a wireless connection between the camera controller 120 and the computer 130 is also possible. In the illustrated implementation, the computer 130 can provide an alternate or supplemental display device. In some implementations, the user (and optionally, a patient) can view the images from the intraoral camera 110 on a display connected to the computer 130. The computer 130 can also serve as an interface to project images in a group display setting, as described in more detail below.

The computer 130 can have one or more software applications to allow for image manipulation. In some implementations, such an application may allow for overlaying images from other sources (e.g., CBCT or scanned models of dentition in video) and other image handling functions. The computer 130 can also provide storage for image data from the intraoral camera 110 and other sources, as well as other data. In some implementations, the computer 130 can perform some or all of the functions of the camera controller 120, such as communicating, manipulating, and/or analyzing data relating to images, accelerometer data, ambient light transmission, etc.

In the dental vision system 100, there is a head-worn display 140 that can selectively display images from the intraoral camera 110, as well as other visual images, to the user. As will be described in more detail below, there may be one or more viewing areas for displaying images for each of two lenses on the head-worn display. The viewing areas can be user-selectable, such that the user can change the location, shape and size of the viewing areas, among other attributes. In the illustrated implementation, the head-worn display is connected to the computer 130 by a connection 132, such as a wireless connection, but a wired connection could also be used. Also, in other embodiments it may be possible for the head-worn display to be connected to the intraoral camera 110 and/or the camera controller 120.

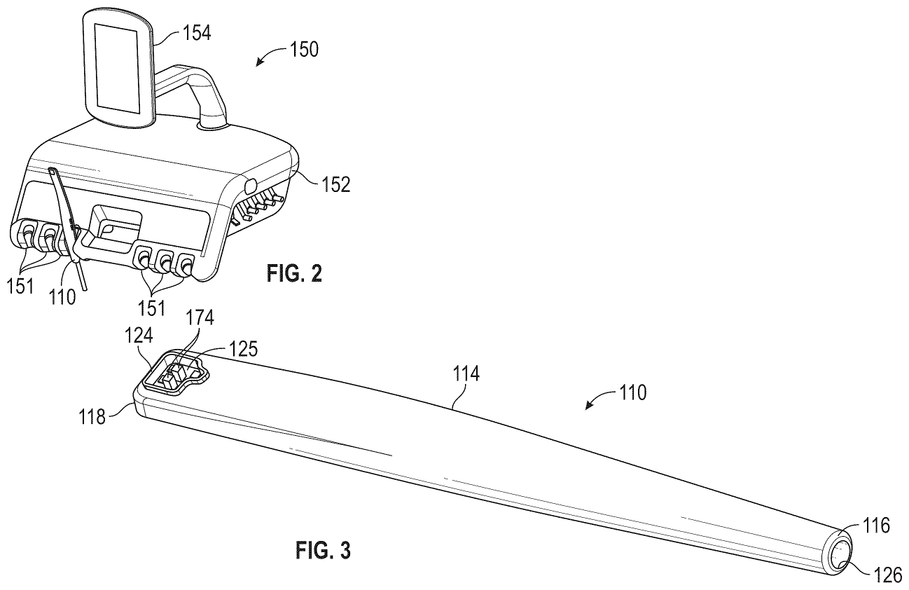

As shown in FIGS. 2-4, the intraoral camera 110 has a generally elongate shape, similar to a stalk or a wand. As shown in FIG. 2, the intraoral camera 110 may be configured to fit in one of multiple handpiece storage positions 151 provided on a housing 152 of a delivery system control head 150. Such a control head 150 may also have a display 154 as shown, which may be one of several displays with which the intraoral camera 110 can selectively interact during use.

The intraoral camera 110 has a body 114 suitably shaped for holding by a user and having a proximal end 116 and a distal end 118. Near the distal end 118, an opening 124 in the housing is defined. A transparent lens 125 (or window) is installed in the opening 124. At the proximal end 116, there is an opening 126 defined for the cable connection 112.

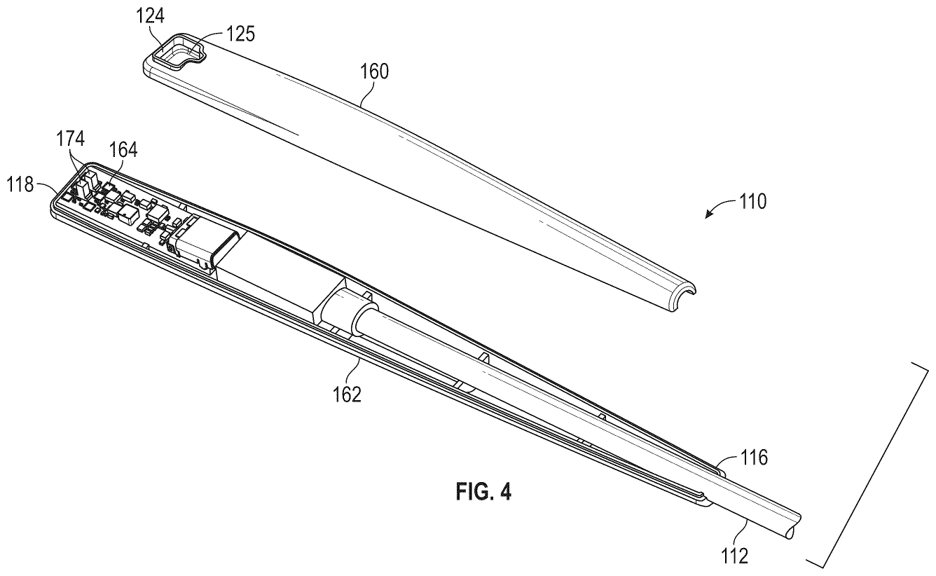

FIG. 4 is an assembly view of the intraoral camera 110 showing an upper housing 160 removed from a lower housing 162. Within the lower housing 162, there is a circuit board component 164 positioned near the distal end 118. The cable connection 112 extends to and has connections with the circuit board component 164. For example, the cable connection 112 can supply power to the circuit board component 164, as well as transfer data and signals to and from the circuit board component 164.

The intraoral camera 110 has at least one camera module, and typically, multiple camera modules, which are typically mounted to one or more circuit boards. For example, in the illustrated implementation, there are two camera modules 174 positioned side-by-side that are mounted to the circuit board component 164. As shown in FIGS. 3 and 4, the camera modules 174 are positioned in alignment with the opening 124 in the upper housing 160. The lens 125 prevents particulates and liquids from entering the housing through the opening 124. The openings 124 and 126, as well as the upper housing 160 and lower housing 162, can be sealed around their edges to protect against moisture entering the body 114 of the intraoral camera 110.

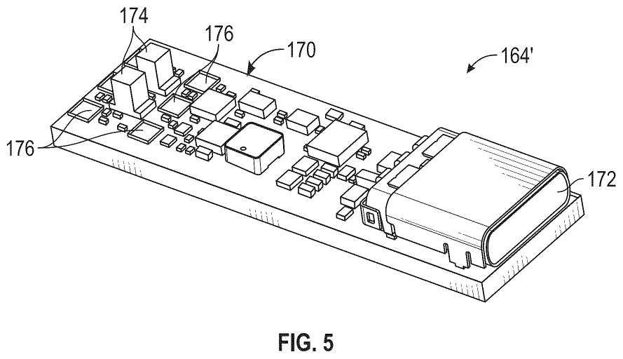

A circuit board component 164′, which is a working prototype similar in some respects to the circuit board component 164 of FIG. 4, is shown in isolation and partially in schematic form in FIG. 5. In addition to the camera modules 174, the circuit board component 164′ has multiple LEDs 176 positioned around the camera modules 174 to provide illumination of the scene. In addition, the circuit board component 164′ has at least one motion sensing accelerometer. Other circuit components of circuits for the intraoral camera 110 are also mounted to the circuit board component. Using a single circuit board component 164 or 164′ for the mounting the camera modules 174 and the LEDs 176 reduces complexity and cost in the design.

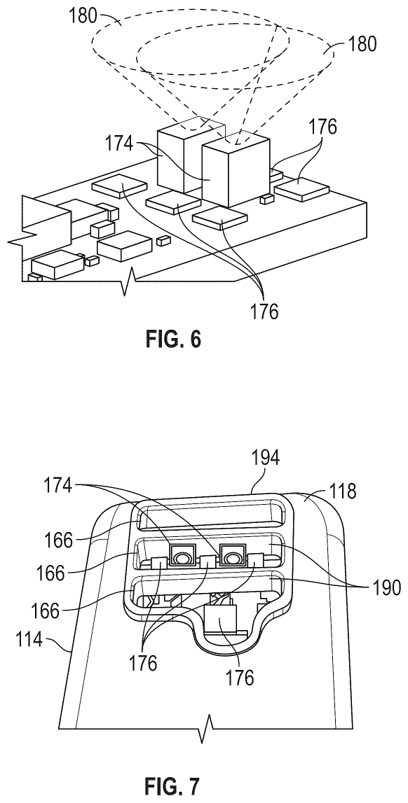

A connector 172 connects the circuit board component 164′ to the connection 112 for supplying electrical power to the circuit board component 164′ and sending data signals to and receiving data signals from the circuit board component 164′. In other implementations, a detachable connector (not shown) can be provided at the proximal end 116 to detachably connect the intraoral camera 110 to the connection 112. In specific implementations, the camera modules 174, also sometimes referred to as cameras, may be CMOS video cameras with an integral lens system of f 4.37 mounted on a common plane with a field of view that overlaps at a distance from the aperture of each camera at 4 mm or less. FIG. 6 is a perspective view of a portion the circuit board component 164, shown schematically, illustrating that the camera modules 174 each have a field of view (FoV) 180 of 104° or more that overlaps with the other.

The opening 124 in the housing may be a single larger, opening as shown in FIGS. 3, 4 and 8. Alternatively, multiple smaller, openings (also referred to as apertures) may be defined, as shown in FIGS. 7 and 9. FIG. 7 shows a distal end 118 having three apertures 166 (slightly spaced from an outer edge 194) defined by two baffles 190 and the surrounding opening. The camera modules 174 and the LEDs 176 are positioned behind, i.e., recessed from, the outermost surface(s) of the apertures 166. Advantageously, the surfaces that define the apertures can extend inwardly past the elevation of the lens surfaces of the camera modules 174 and/or the light emitting surfaces of the LEDs 176 to more effectively block stray light from the LEDs from reaching the lens surfaces of the camera modules. As stated, the apertures 166 are covered by one or more clear covers or lenses, such as the lens 125 (see, e.g., FIG. 3).

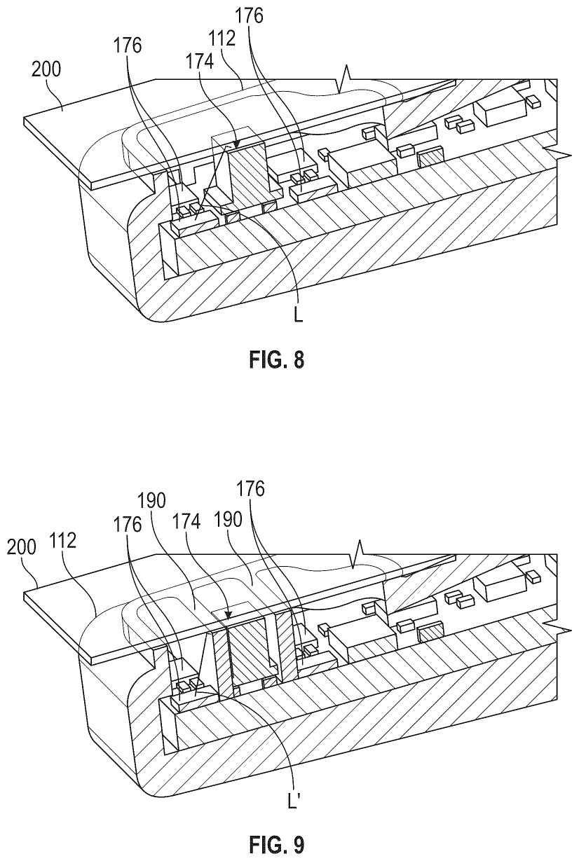

To control against cross-contamination of potentially infectious material between patients, the intraoral camera 110 can be fitted with a barrier sheath 200, which is shown partially in FIGS. 8 and 9, shaped to fit over at least the distal end 118. A portion of the sheath 200 overlying the apertures 166 is highly transparent to minimize distortion or other degradation of the image received by the camera modules 174. The barrier sheath 200 can be designed as a single-use sheath or a multiple-use sheath. In some conventional intraoral cameras, the barrier sheath rests on LEDs that protrude from the housing, thereby minimizing some reflected light and glare effects, but such designs require separate circuit boards for the LEDs and camera elements to allow each to be at a different elevation, which add complexity and cost.

As discussed above, in the intraoral camera 110, the baffles 190, also referred to as optical barriers, are configured to block stray light. The baffles 190 can be formed into the housing or provided as separate elements. The baffles 190 are configured to be optically opaque. The outer extents of the baffles 190 protrude beyond the LEDs 176 and the camera modules 174 (i.e., the baffles are higher in elevation). Without the baffles 190 or a similar structure, a portion of the light emitted from the LEDs 176 can reflect off an inner surface of the sheath 200 and impinge on the camera modules 174 (see typical ray L in FIG. 8), causing undesired glare in the resulting image.

Alternatively, as shown in FIG. 9, the sheath 200 and the intraoral camera 110 can be configured such that when the sheath 200 is installed on the intraoral camera 110, it fits tautly on the top of the baffles 190, ensuring that there is no gap between the sheath 200 and the top of the baffles 190 through which light might penetrate and reach the camera modules 174. Thus, a typical ray L′ emitted from the LEDs that reflects off the inner surface of the sheath 200 now strikes the nearest baffle 190 and is attenuated by its opaque surface. The line of sight of the camera modules 174 is thus blocked by the wall of the nearest baffle 190.

The dental vision system 100 can be configured to provide real-time image stabilization for the intraoral camera 110. For example, an image stabilization function programmed in the operating system for the dental vision system 100, which can reside in a CPU, camera controller and/or other device controller, can be selected in system settings. The image stabilization function mitigates the effects of involuntary motion in the user's hand(s) as the intraoral camera 110 is held and moved, which tends to produce a clearer image by discarding spurious effects. The image stabilization function is carried out by a CPU or controller using inputs received from the motion sensing accelerometer 170 or other similar motion data. Repetitive motion in any axis that is low in magnitude and regular is mitigated. Examples include jitter, defined as very small displacements in frequencies between 1 Hz and 60 Hz with an amplitude greater than one pixel in the horizontal, vertical or combined horizontal and vertical extents and blur, defined as image motion at a rate of change less than 1 Hz with an amplitude greater than one pixel. A clock marks the video frames and correlates the time of the start of the motion, the direction of the motion and the rate of change of the motion to the video frames. Corrections are calculated and applied to each frame. In some implementations, the motion sensing accelerometer 170 is mounted on a plane common with one or both of the camera modules 174.

In addition to image stabilization, the CPU or controller can also be programmed to display images in an appropriate orientation even as the intraoral camera 110 is translated or rotated. For example, images can be automatically rotated as appropriate such that a specific orientation is maintained during a sequence. A reference direction, such as a “gravitational up” direction or another user-selected direction can be specified. Alternatively, a magnetic field sensor can be used to define a three-dimensional coordinate system. The video image can then be rotated according to a user-selected principal direction in that coordinate system.

The auto-rotation of images shares some similarities with auto-rotation of a smart phone or tablet screen, but there are several key differences. The auto-rotation of images is based on the orientation of the camera, not the orientation of the display device. Also, the user of the present system can select the reference direction to be used in determining the degree of rotation. For example, the direction of earth's gravitational field can be chosen to define the downward direction, and the displayed image or graphics can be rotated accordingly based on that direction. Alternatively, the user can select the principal axis to be aligned in an alternative direction, e.g., the major axis of the patient's head or the direction of the patient's mandible, a specific tooth or other feature of interest.

Moreover, the auto-rotation of images as implemented in the intraoral camera is substantially continuous, with rotation by a small incremental angle producing a similar rotation in the displayed image in the opposite direction, thus maintaining the orientation of the displayed image relative to the predetermined principal axis. In contrast, auto-rotation for a smart phone is typically limited to 90 degree increments (as the smart phone is rotated, the displayed image remains in a fixed orientation until the rotation exceeds a threshold angle, at which point the image rotates by 90 degrees).

In some implementations, the intraoral camera has a compass instead of or in addition to an accelerometer. Using the compass or the combination of the accelerometer and the compass provides for detecting the orientation of the device when the principal axis is to be aligned with a direction other earth's gravitational field. For example, with a patient in a supine position, the user may set up auto-rotation aligned with the major axis of the patient's head and the plane of the camera sensing surface parallel to the floor. Auto-rotation under these conditions is not possible with an accelerometer only, but is possible with the compass or combination of the compass and the accelerometer.

As used herein, the phrase “at least substantially,” when modifying a degree or relationship, includes not only the recited “substantial” degree or relationship, but also the full extent of the recited degree or relationship. A substantial amount of a recited degree or relationship may include at least 75% of the recited degree or relationship. For example, a first direction (or orientation) that is at least substantially parallel to a second direction includes a first direction that is within an angular deviation of 22.5° relative to the second direction and also includes a first direction that is identical to the second direction.

The head-worn display 140 can have a spectacle configuration as shown, or any alternative form, e.g., goggles, face shield, helmet and eye shield, etc. The portion of the head-worn display associated with producing the visual images can be built into the head-worn apparatus or be a separate module that attaches to the head-worn support apparatus, the latter implementation allowing greater flexibility to the user to select eyewear of their choosing to use in combination with the video display module. The term “head-worn display” includes all of the aforementioned configurations. In the head-worn display 140, there are a pair of lenses (such as right and left lenses 142R, 142L, respectively) or lens areas (if only a single larger lens is used). At least one but typically both of the lenses 142R, 142L have respective display areas 144R, 144L facing the wearer that are user-selectable to display images and other content. The display areas 144R, 144L may fill the total viewing area of the right and left lenses, respectively, or they may fill just a portion of the total viewing area of each lens. The display areas 144R, 144L may be the same in size and position within the respective lens 142R, 142L, or they may differ in size and/or position. The size and position of the display areas may be static or selectable by the wearer. In a specific implementation, it is possible to have display areas that are separately sized and positioned, but user eyestrain is lessened if the display areas are approximately the same in size and relative position within each eye's field of view. Optionally, there may be more than one display area for one or both of the lenses 142R, 142L.

In the illustrated implementation, there are non-display areas 146R, 146L bordering the display areas 144R, 144L. The non-display areas 146R, 146L may be transparent, or may have an optical gradation from transparent to opaque (which can be fixed or changeable).

The display areas 144R, 144L can also be configured in optical gradations from transparent to opaque. When an image is being displayed in the display area, then it is preferable to have the display area portion of the lens configured as fully opaque to reduce light transmission through this area and improve contrast for viewing the image. Conversely, when an image is not being displayed in the image area, then it is preferable for the display portion to be fully transparent to allow the wearer to have full field of view through the lens.

To implement changeable optical gradations, liquid crystal technology can be used. In addition, opaque materials and/or coatings can be used to configure any portion of the lens as an opaque portion.

In the illustrated implementation of the intraoral camera 110 having two camera modules 174, the image from the leftmost of the camera modules 174 is displayed in the display area 144L of the left lens 142L, and the image from rightmost of the camera modules 174 is displayed in the display area 144R of the right lens 142R. Other options are also possible, such as only displaying one image, with the other display area being configured for direct through viewing by the wearer, or viewing of other content (e.g., comparison image data from the patient's record), subject to user preference. As described above, the display area 144L and/or the display area 144R could be enlarged to the full size of the respective left lens 142L and/or right lens 142R to substantially fill the user's field of view with the displayed image.

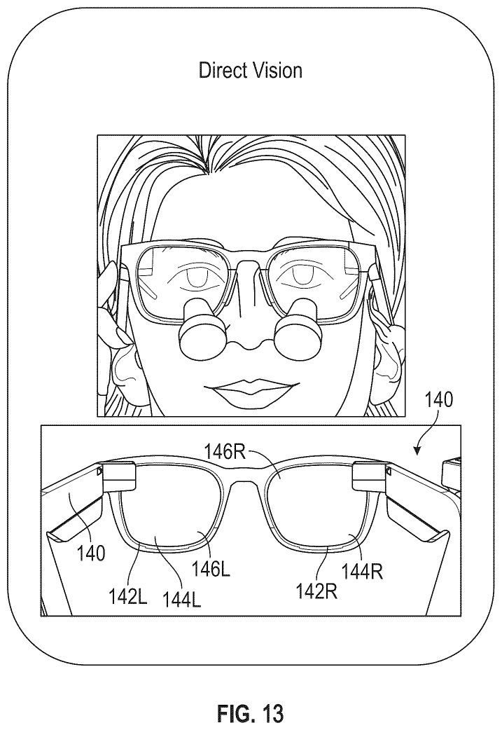

FIG. 13 illustrates the head-worn display 140 configured in a direct vision mode in which the display areas 144R, 144L are configured as transparent areas similar to the non-display areas 146R, 146L to provide the wearer with uninterrupted direct vision through the lenses 142R, 142L. As illustrated in the upper portion of the figure, the direct vision mode is compatible with allowing the wearer to use direct vision through optional optical loupes without distracting borders and content.

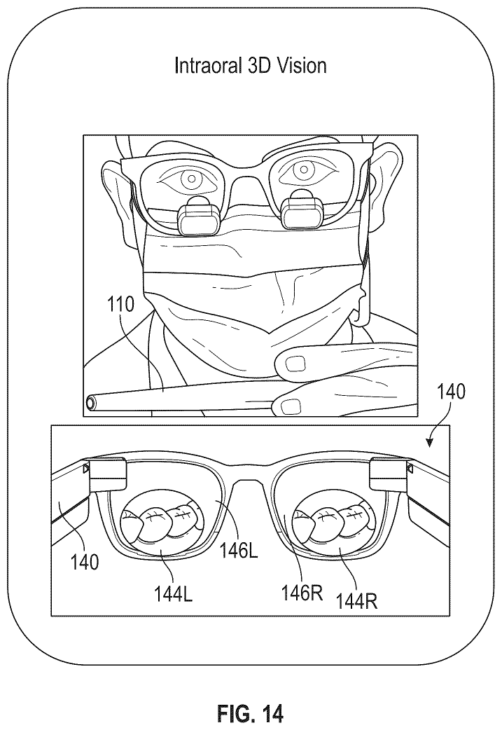

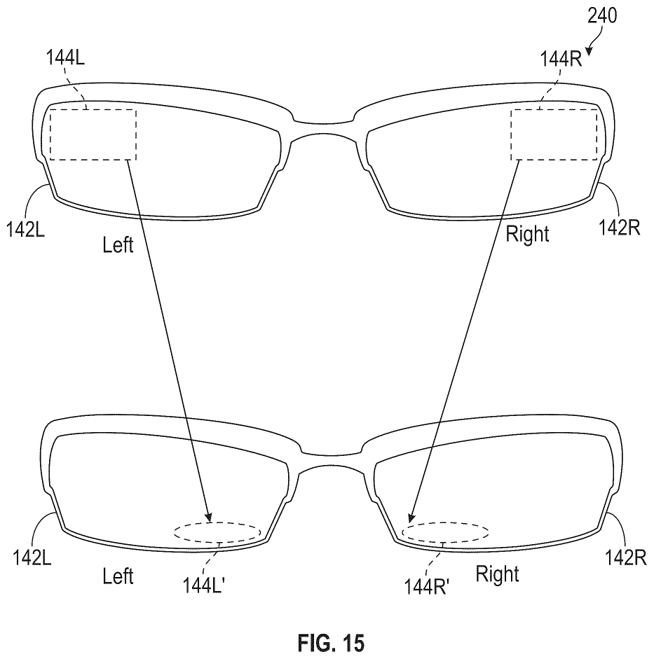

FIG. 14 illustrates the head-worn display 140 configured in an intraoral camera mode to display images from the intraoral camera 110 in both display areas 144R, 144L, such as in a 3D format or another suitable format. In a 3D format, images of the same subject taken from two slightly different positions are displayed, one image to each eye, to enable parallax, which provides 3D perception. FIG. 15 shows that an image from the leftmost of the camera modules 174 is displayed in the display area 144L and an image from the rightmost of the camera modules 174, i.e., at a slightly different position than the leftmost of the camera modules, is displayed in the display area 144R. In FIGS. 13 and 14, the users are shown wearing head-worn displays having optional loupes that provide magnification, but such loupes are not required for intraoral camera operation.

FIG. 15 is a schematic view of the head-worn display 240 showing the left lens 142L with the display area 144L and the right lens 142R with the display area 144R. As indicated in the drawing, the user has changed the display areas 144L, 144R, which have a rectangular shape and are positioned in an upper outer position, to the display areas 144L′, 144R′ that have an elliptical shape and are positioned in an inner lower position. It would also be possible to have a horizontally split screen with upper and lower display areas within each lens, such as to show the camera images in one set of the display areas and the view from the loupes in the other set of display areas.

It is noted that one or more display areas may be located at positions other than on the inner surface of a lens, including, e.g., at a location between the lens and the user's eye.

In any of the examples described herein, the described images can be still images or video images, can be from any camera (the intraoral camera 110, a camera on the head-worn display or another source), and can be selectively magnified. Also, the images can be in the visible spectrum, or outside the visible spectrum, such as in situations where it is helpful to detect fluorescence. As described above, in some implementations, the lenses 142L, 142R are split horizontally to define display areas (which can be the upper areas or the lower areas) and normal viewing areas (i.e., the other of the upper areas and the lower areas).

Optionally, the head-worn display 240 can be configured to allow the user to move the front portion out of the way of their view, such as by flipping up the front portion to a raised position (not shown). Also, the user can simply remove the head worn display when they desire to view a scene unimpeded by the head worn display.

The gradation can be adjusted in one or more of the following ways: (a) by a physical button or other control within the head-worn display, (b) by a keypress on the keyboard of the control computer or other input, (c) by a control or gesture on the computer screen actuated by a user input, (d) by a unique motion of the wearer's head, (e) by voice command, (f) by a control display on the head-worn device screen or (g) another suitable input.

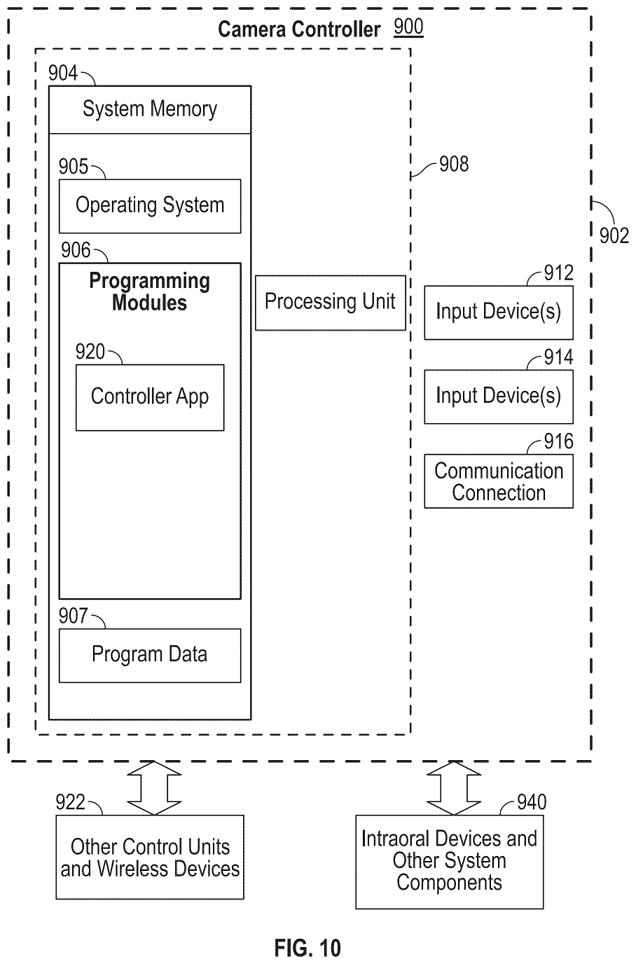

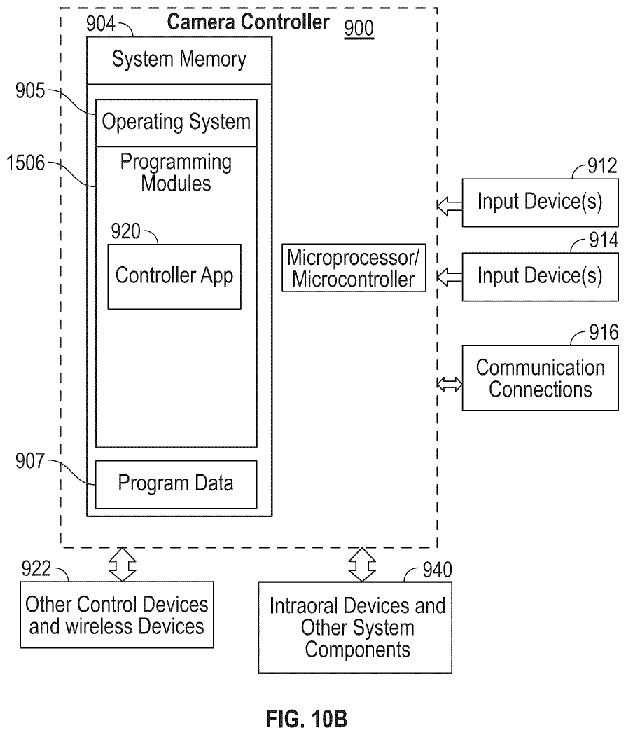

FIG. 10 is a block diagram of a camera controller 900 implemented in a computing device 902 separate from the intraoral camera 110. FIG. 10B is a block diagram of the camera controller 900 implemented as a standalone camera controller.

Consistent with implementations of the system, memory storage and processing functionality may be implemented in a computing device, such as the camera controller 900. Any suitable combination of hardware, software, or firmware may be used to implement the memory storage and processing unit. For example, the memory storage and processing unit may be implemented with the camera controller 900 or any other control unit and wireless devices 922, in combination with the camera controller 900. The aforementioned system, device, and processors are examples and other systems, devices, and processors may comprise the aforementioned memory storage and processing unit, consistent with embodiments of the disclosure.

In further aspects, a system consistent with an embodiment of the disclosure may include a computing device, such as the controller 900. In a basic configuration, the controller 900 may include at least one processing unit (or microprocessor or microcontroller) and a system memory 904. Depending on the configuration and type of computing device, the system memory 904 may comprise, but is not limited to, volatile (e.g., random-access memory (RAM)), non-volatile (e.g., read-only memory (ROM)), flash memory, or any combination thereof. The system memory 904 may include an operating system 905, one or more programming modules 906, and may include program data 907. The operating system 905, for example, may be suitable for controlling operation of the controller 900. In one embodiment, the programming modules 906 (see also 1506 in FIG. 10B) may include a controller application (“app”) 920. Embodiments of the disclosure may be practiced in conjunction with a graphics library, other operating systems or any other application program, and are not limited to any particular application or system. This basic configuration is illustrated in FIG. 10 by those components within a dashed line 908.

Advantageously, the app may provide a user with information as well as serve as the user interface to operating the embodiment of the invention. The app may include one or more graphic user interfaces (GUIs). The app can be resident on a display system controller, the camera controller, a standalone computer, a mobile device (e.g., a smart phone or tablet) or another suitable device configured to communicate with the system.

Among the GUIs of the app may be a GUI allowing the user to pick which, if there is more than one, imaging device and/or lighting elements to activate, and to select (if available) one or more operating parameters or characteristics (such as intensity or lighting type) of the device(s). The user may be able to adjust such selections without having to deactivate the embodiment from a GUI of the app. The user may also use the app to turn on and turn off the device components. Another advantage of the app is that the app may present the user with a GUI that depicts the patient's mouth and/or teeth (or a generic mouth/teeth) and show where the illumination and/or camera field of view are being applied. The GUI may include additional or other information relating to the image data being captured and may also present the user with information received from the device components, and or diagnostic information.

Controller 900 may have additional features or functionality. For example, controller 900 may also include additional data storage devices (removable and/or non-removable) such as, for example, magnetic disks, optical disks, or tape. Computer storage media may include volatile and nonvolatile, removable and non-removable media implemented in any method or technology for storage of information, such as computer readable instructions, data structures, program modules, or other data. System memory 904 is an example of computer storage media (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by controller 900. Any such computer storage media may be part of controller 900. Controller 900 may also be operative with input device(s) 912 such as a keyboard, a mouse, a pen, a sound input device, a touch input device, etc. Input device(s) 912 may be used to, for example, manually access and program controller 900. Output device(s) 914 such as a display, speakers, a printer, etc. may also be included. These devices are examples only, and others may be used.

Controller 900 may also contain a communication connection 916 that may allow it to communicate with other control units and wireless devices 922. In addition, the controller 900 can be linked to other devices and system components 940 (including display devices, etc.), which include the intraoral camera 110 for the FIG. 10 implementation, such as over an encrypted network in a distributed computing environment. Communication connection 916 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, Bluetooth, radio frequency (RF), infrared, and other wireless media. The term computer readable media as used herein may include both storage media and communication media.

As stated above, a number of program modules and data files may be stored in the system memory 904, including the operating system 905. While executing on the processing unit 902, the programming modules 906 (e.g., the intraoral visualization device controller application 920) may perform processes including, for example, one or more of stages or portions of stages of the method as described above. The app 920 may be configured to operate the device components and receive instructions from, for example, the communications connection module 916. This is an exemplary only, and the processing unit 902 may perform other processes.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

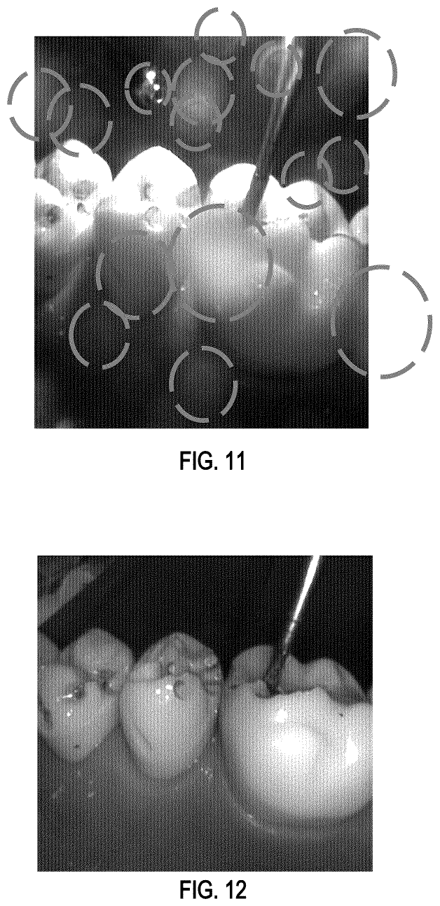

FIG. 11 is an example image from one or more of the camera modules 174 in which droplets of spray and spatter from patient's the oral cavity are present in the image as shown by the circled areas. The droplets, which are considered transient artifacts, tend to obscure parts of the image, and thus it is desirable to remove the droplets or mitigate their effect on image quality. FIG. 12 shows an image corresponding to FIG. 11 after real-time video software enhancement has been applied to remove or at least reduce the effects of the transient artifacts.

Various approaches can be used to remove transient artifacts, including but not limited to droplets of water, saliva and blood, or particles of tooth or filling materials, from video images. In some approaches, an image processor is used. One approach involves mapping the red, green and blue (RGB) values of individual pixels that comprise a video frame; storing the map in short-term, dynamic memory along with the maps for optionally one to several of the most recent preceding frames; identifying features in the map (i.e., groups of adjacent pixels occurring in sequential frames which have similar spatial distribution of RGB values but which may move in a progressive fashion from frame to frame); determining the speed, direction, appearance and disappearance of features; determining features which are artifacts (i.e., features which appear, disappear or move with a different speed or direction compared to the majority of features associated with the subject); and replacing the artifact features by substituting the RGB values of the pixels designated as artifact features with the RGB values of the subject, determined from preceding frames or estimated based on adjacent pixels in the current frame. Such video enhancement must be performed at a rate consistent with the frame rate of the camera and display system, which is preferably at least 30 frames per second to avoid observable latency of the displayed video stream. At a frame rate of 30 frames per second, each frame must be processed in less than about 0.033 seconds.

Artificial intelligence can be applied to facilitate or enhance the execution of artifact removal, for example by identifying the type of feature in the image (e.g., tooth, gum, instrument bur, droplet) based on patterns within a frame and using that information to streamline the determination of which features are artifacts and how to replace them.

Eye tracking, including mobile eye tracking implemented with the head-worn display (i.e., head-mounted eye tracking), can be used to receive inputs from the wearer to specify responses, commands, settings, etc. Inputs can be generated from position of the eye (retina, pupil, other), rate of movement of the eye and/or dwell of the eye. The head-worn display 140 can be fitted with an eye camera(s) or mirror(s) for one eye (monocular) or both eyes (binocular), as well as a front-facing scene camera(s) that records the scene or field of view. It is also possible to use augmented reality (AR) and virtual reality (VR) techniques and devices to implement the ability of the wearer to control the intraoral camera, including specifying the types of image data displayed to the wearer and the positions thereof. Similarly, voice command and/or hand gesture technologies can also be used.

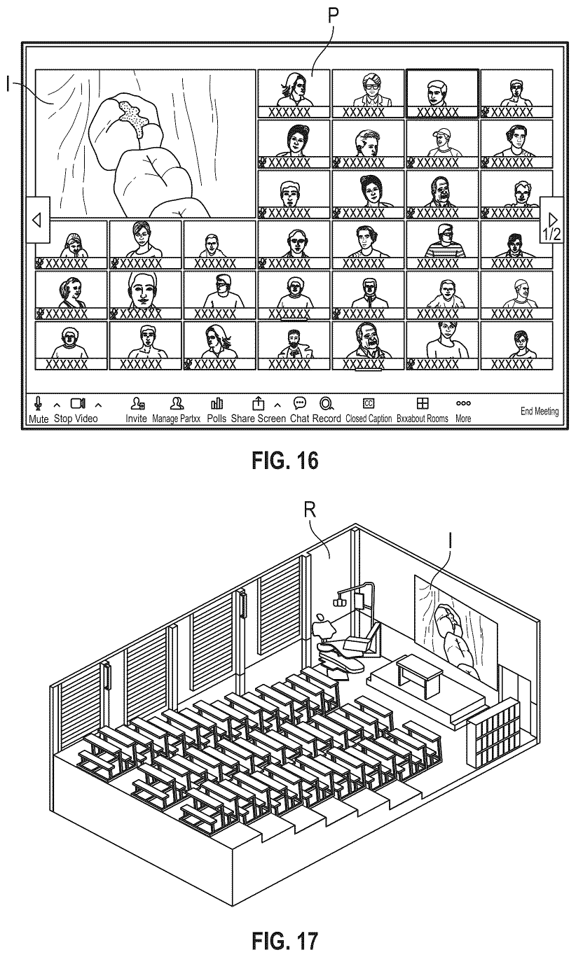

The intraoral camera 110 image information can be used by the practitioner (dentist, dental hygienist, other) as described above, shared with the patient (e.g., to explain treatment steps or options), as well as shared with others. For example, the image information from the intraoral camera 110 can be shared with others in a teaching or continuing education setting. FIG. 16 is a representative screen display of a video conference call in which a screen having intraoral camera image data I from a patient is being shared with participants P for educational purposes. It would also be possible to use the same video conference model to enable professional consulting about a patient's case or to carry out training. FIG. 17 shows an example of learning facility R with seating for an audience, an operatory for an instructor to carry out a dental procedure and a screen on which to display intraoral camera image data I from the procedure (as well as other information) to the in-person audience and on-line audience.

In a similar way, image data can be shared with others to provide for remote troubleshooting and/or service of equipment, instruments and other devices in a dental practice. The head-worn display 140 can be used to acquire image data of equipment for which technical support is needed. Image data showing the equipment and the technical issue can be shared with the technical support provider, such as in a video conference. The technical support provider can use the image data to confirm the technical issue and to seek additional details to help resolve the issue. The technical service provider can also supply image data, such as graphics showing the equipment and how to service it, which can be displayed for the wearer in the head-worn display to make the steps easier to follow and execute. A system using AI (artificial intelligence) can help match image data to identify components, assemblies, supplies, etc., such as for troubleshooting, service, replacement and the like.

Artificial intelligence (AI), as well as augmented intelligence, can be incorporated to improve the functionality of the system in other ways, as well. AI and augmented intelligence can be used as described above in virtual reality and augmented reality applications for treatment visualization and patient education. Additionally, AI/augmented intelligence algorithms can be used to analyze dental images, such as radiographs and intraoral scans, to assist dentists in detecting and diagnosing oral diseases with greater accuracy and efficiency. Also, AI/augmented intelligence-based software can assist dentists in treatment planning by analyzing patient data, case histories and treatment outcomes. These tools provide valuable insights, helping dentists make informed decisions about treatment options, materials and techniques. Further, AI and augmented intelligence algorithms can be used for data analysis and predictive analytics. This technology aids in early detection, leading to timely interventions and improved patient outcomes.

Any computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Perl, JavaScript, Adobe Flash, or any other suitable programming language. Likewise, the disclosed technology is not limited to any particular computer or type of hardware. Certain details of suitable computers and hardware are well known and need not be set forth in detail in this disclosure.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the internet, an intranet, software applications, cable (including fiber optic cable), magnetic communications, electromagnetic communications (including RF, microwave, and infrared communications), electronic communications, or other such communication means.

The disclosed methods, apparatus, and systems should not be construed as limiting in any way. Instead, the present disclosure is directed toward all novel and nonobvious features and aspects of the various disclosed embodiments, alone and in various combinations and sub combinations with one another. The disclosed methods, apparatus, and systems are not limited to any specific aspect or feature or combination thereof, nor do the disclosed embodiments require that any one or more specific advantages be present or problems be solved.

In view of the many possible embodiments to which the disclosed principles may be applied, it should be recognized that the illustrated embodiments are only preferred examples and should not be taken as limiting in scope. Rather, the scope of protection s defined by the following claims. We therefore claim all that comes within the scope and spirit of these claims.

What is claimed is:

1. A dental vision system, comprising:
an intraoral camera component positionable within an oral cavity of a patient and movable manually by a user to obtain patient image data from various viewpoints within the oral cavity, the intraoral camera component having at least two camera modules and an inertial accelerometer configured to collect position and movement data relating to the intraoral camera component; and
a camera controller linked to the intraoral camera component, wherein the camera controller receives the patient image data and the position and movement data relating to the intraoral camera component and is configured to determine a selected viewing orientation for the patient image data based at least in part on the position and movement data.

2. The dental vision system of claim 1, wherein the camera controller is programmed to apply an angular rotation to the patient image data such that each patient image is substantially aligned with the selected viewing orientation.

3. The dental vision system of claim 1, wherein the selected viewing orientation comprises a gravitational up orientation.

4. The dental vision system of claim 1, wherein the selected viewing orientation is determined with reference to a magnetic compass.

5. The dental vision system of claim 1, wherein the selected viewing orientation is determined with reference to an absolute three-dimensional coordinate system.

6. A dental vision system, comprising:
an intraoral camera component positionable within an oral cavity of a patient, the intraoral camera component comprising
at least two video camera modules that are spaced apart from each other having respective fields of view that overlap each other,
a plurality of LEDs positioned around the at least two video camera modules, and
at least one baffle positioned between one of the at least two video camera modules and a respective outwardly adjacent group of the plurality of LEDs, the at least one baffle comprising a wall extending outwardly to reduce unwanted optical effects in images obtained by the at least two video camera modules.

7. The dental vision system of claim 6, wherein the at least one baffle extends outwardly to a baffle height higher than a height of the one of the at least two video camera modules.

8. The dental vision system of claim 6, wherein the at least one baffle has a distal end, and wherein the distal end is configured to support a transparent barrier film applied to cover the at least two video camera modules, wherein the wall of the at least one baffle is configured to block light rays emanating from the outwardly adjacent group of LEDs and reflected from the transparent barrier film to prevent the light rays from interfering with the images taken by the video camera modules.

9. The dental vision system of claim 1, wherein the at least two camera modules are mounted to a common plane2.

10. A dental vision system, comprising:
an intraoral camera component positionable within an oral cavity of a patient and movable manually by a user to obtain patient image data from various viewpoints within the oral cavity; and
a head-worn display with at least one viewing area configured to selectively display the patient image data from the intraoral camera component to a wearer of the head-worn display, wherein a location and size of the patient image data to be displayed are user selectable to improve visualization of the patient image data by the wearer.

11. The dental vision system of claim 10, wherein optical gradation in the viewing area can be reconfigured in multiple steps from opaque to transparent.

12. The dental vision system of claim 10, wherein the head-worn display comprises a liquid crystal film selectively changeable to change optical gradation in the viewing area.

13. The dental vision system of claim 12, wherein the liquid crystal film is within or applied to a lens of the head-worn display.

14. The dental vision system of claim 10, wherein the viewing area of the head-worn display is configurable to change an optical gradation in the viewing area to improve visualization of the patient image data by the wearer.

15. The dental vision system of claim 10, wherein the head-worn display comprises a spectacle lens pair including a first spectacle lens and a second spectacle lens, and wherein the at least one viewing area comprises a portion of a total viewing area of at least one of first spectacle lens and the second spectacle lens.

16. The dental vision system of claim 15, wherein at least a portion of the spectacle lens pair comprises a direct vision viewing area that is not configured to selectively display the patient image data.

17. The dental vision system of claim 10, further comprising a camera controller linked to the intraoral camera component and the head-worn display, wherein the camera controller receives the patient image data and is configured to transmit the patient image data wirelessly to the head-worn display.

18. The dental vision system of claim 10, wherein the head-worn display is configured to display 3D patient image data.

19. A dental vision system, comprising:
an intraoral camera component positionable within an oral cavity of a patient and movable manually by a user to obtain patient image data from various viewpoints within the oral cavity; and
a head-worn display with at least one viewing area configured to selectively display the patient image data from the intraoral camera component, further comprising a pupil tracking module configured to track at least one pupil of a wearer of the head-worn display to assist in controlling display of the patient image data on the at least one viewing area.

20. The dental vision system of claim 19, wherein the pupil tracking module is configured to track a position of the at least one pupil of the wearer.

21. The dental vision system of claim 19, wherein the pupil tracking module is configured to track a rate of movement of the at least one pupil of the wearer.

22. The dental vision system of claim 19, wherein the pupil tracking module is configured to track dwell of the at least one pupil of the wearer that exceeds a predetermined time.

23. A dental vision system, comprising:
an intraoral camera component positionable within an oral cavity of a patient and movable manually by a user to obtain patient video image data from various viewpoints within the oral cavity; and
an image stabilizer configured to process the patient video image data in raw form and generate stabilized patient video image data to reduce noticeable motion in the patient video image data.

24. The dental vision system of claim 23, wherein the image stabilizer is configured to address jitter in the patient video image data, including small displacements in frequency from 1 Hz to 60 Hz with a dimensional amplitude greater than one pixel in a horizontal extent, a vertical extent or combined horizontal and vertical extents.

25. The dental vision system of claim 23, wherein the image stabilizer is configured to address blur in the patient video image data, including small displacements at a rate of change of less than 11 Hz with an amplitude of greater than one pixel.

26. The dental vision system of claim 23, wherein the intraoral camera component comprises at least one camera module and at least one MEMS accelerometer positioned relative to the at least one camera module to detect 6-axis acceleration of the camera module along translational and rotational axes.

27. The dental vision system of claim 26, further comprising a camera controller, wherein the camera controller receives 6-axis acceleration data in an output signal from the at least one MEMS accelerometer and generates pixel displacement and pixel displacement rate of change values correlated with a clock signal to apply to the patient video image data to produce a corrected patient video image for display.

28. The dental vision system of claim 23, further comprising an image processor configured to remove transient artifacts occurring in the oral cavity substantially in real time to improve image quality.
